(19) United States
(12) Plant Patent Application Publication
(10) Pub. No.: US 2024/0373768 P1
(43) Pub. Date: Nov. 7, 2024

(54) APPLE TREE NAMED 'HOT81A1'

(50) ;

(71) Applicants: The New Zealand Institute for Plant and Food Research Limited, Auckland (NZ); Institut De Recerca Tecnologia Agroalimentaries (IRTA), Barcelona (ES); FruitFuturA.I.E, Lledia (ES)

(72) Inventors: Richard VOLZ, Havelock North (NZ); Cristian FONTICH, Lleida (ES)

(73) Assignees: The New Zealand Institute for Plant and Food Research Limited, Auckland (NZ); Institut De Recerca Tecnologia Agroalimentaries (IRTA), Barcelona (ES); FruitFutur A.I.E, Lledia (ES)

(21) Appl. No.: 18/831,021

(22) Filed: May 1, 2024

Related U.S. Application Data
(60) Provisional application No. 63/464,097, filed on May 4, 2023.

(51) Int. Cl. A01H 6/74

(52) U.S. Cl. CPC A01H 6/7418
USPC PLT/161

(57) ABSTRACT

A new and distinct apple variety is described. The variety results from crossing the apple varieties ‘A121R18T089’ (not patented) and Gala ‘Baigent’ (U.S. Plant Pat. No. 4,121) in Havelock North, New Zealand. The fruit of the apple tree of this new variety have an attractive appearance characterized by its dark red color and creamy white background and it early harvest compared to other selections. This new variety has been named ‘HOT81A1’.

Genus and species plant named: Malus domestica.

Variety denomination: ‘HOT8411’.

### CROSS REFERENCE TO RELATED APPLICATION

This application claims the benefit of U.S. Provisional Application No. 63/464,097, filed May 4, 2023, the disclosure of which is incorporated by reference in its entirety.

### BACKGROUND OF THE INVENTION

The new plant variety described herein resulted from a selection from a population of seedlings derived from the controlled crossing of the apple varieties ‘A121R18T089’ (not patented) (seed parent) and Gala ‘Baigent’ (U.S. Plant Pat. No. 4,121) (pollen parent). The planned cross was made in 2004 on trees located in Havelock North, Hawke's Bay, New Zealand and the seed transported and sown in plots in Gimenells near Lleida, Catalonia, Spain. ‘HOT81A1’ was selected in 2007 as a single plant from among a population of seedlings and was selected for its attractive appearance, good flavor, texture and storage. It was asexually propagated first in Lleida, Spain by budding onto M9 rootstock. The variety has since been observed over three asexually propagated generations and found to remain true to type.

### SUMMARY OF THE INVENTION

‘HOT81A1’ is characterized by its attractive fruit with good texture and flavor, harvesting in late August in Spain. Under growing conditions in Spain ‘HOT81A1’ is distinguished from other apple varieties due to the following unique characteristics: Compared to ‘Baigent’, ‘HOT81A1’ has an earlier harvest season by approximately 2 weeks, and a significantly higher proportion of the skin surface is covered by over color, 70-90%, compared to ‘Baigent’, 40-50%.

Under the growing conditions in Spain the new variety was able to be distinguished from its parents ‘Baigent’ and ‘A121R18T089’, by the following characteristics:

‘Baigent’: The time of eating maturity is later than ‘HOT81A1’.

‘A121R18T089’: ‘HOT81A1’ fruit is larger than ‘A121R18T089’ apples. In addition ‘HOT81A1’ has a fruit skin overcolor pattern of a solid flush with weakly defined stripes; compared to ‘A121R18T089’ which has a solid flush overcolor pattern.

Asexual reproduction of this new variety by budding and grafting shows that the aforementioned characteristics are true to form and are established and transmitted through succeeding propagation. This is true of buds grafted onto M9 rootstocks.

### BRIEF DESCRIPTION OF PHOTOGRAPHS

The accompanying photographs show typical specimens of the tree, foliage and fruit of the new variety as depicted in colors as nearly true as is reasonably possible to make the same in a color illustration of this character.

FIG. 1 shows a 3 year old tree of ‘HOT81A1’ scion on M9 rootstock in the field.

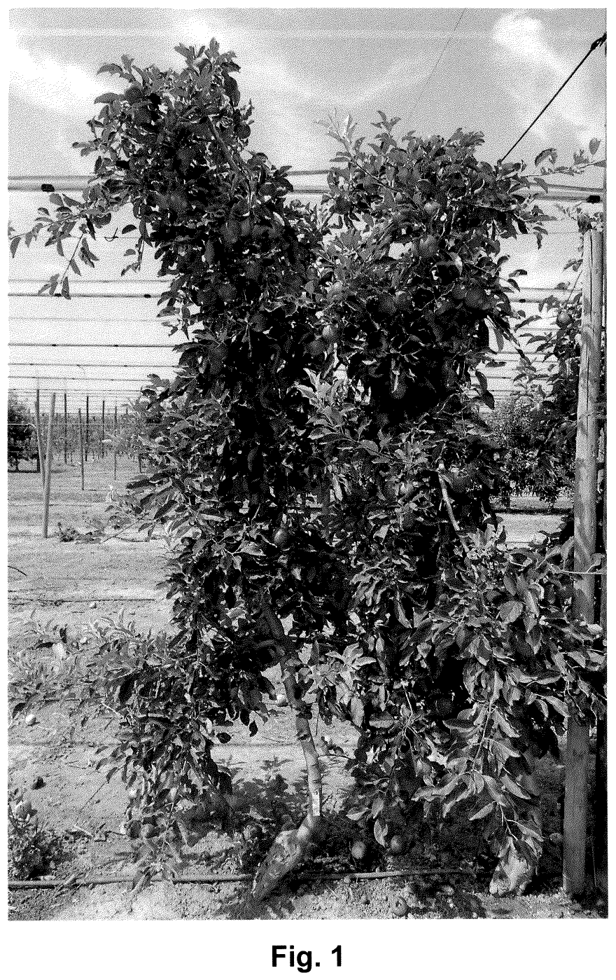

FIG. 2 shows whole ‘HOT81A1’ fruit.

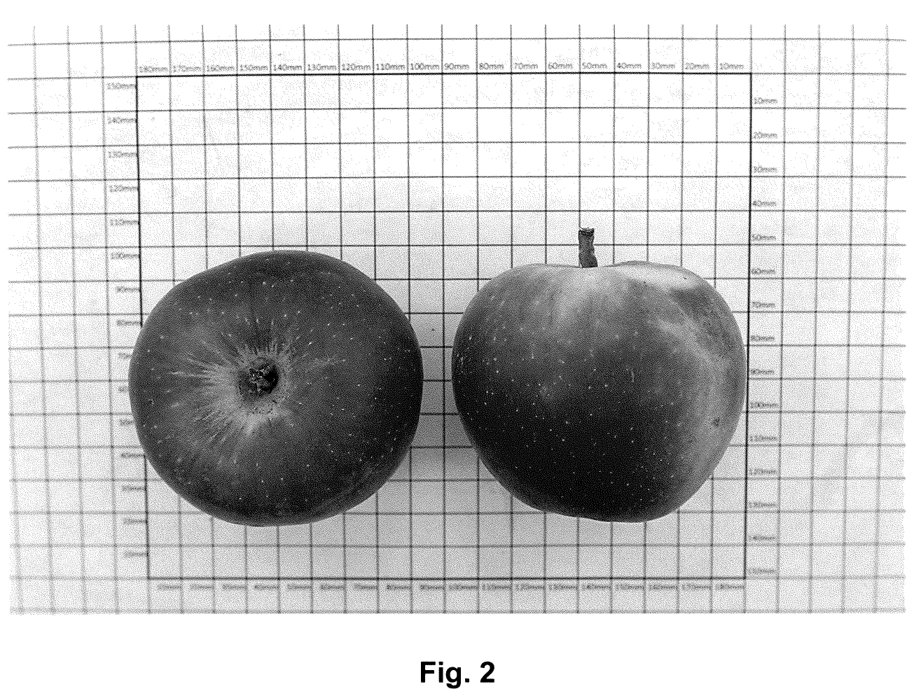

FIG. 3 shows ‘HOT81A1’ flower

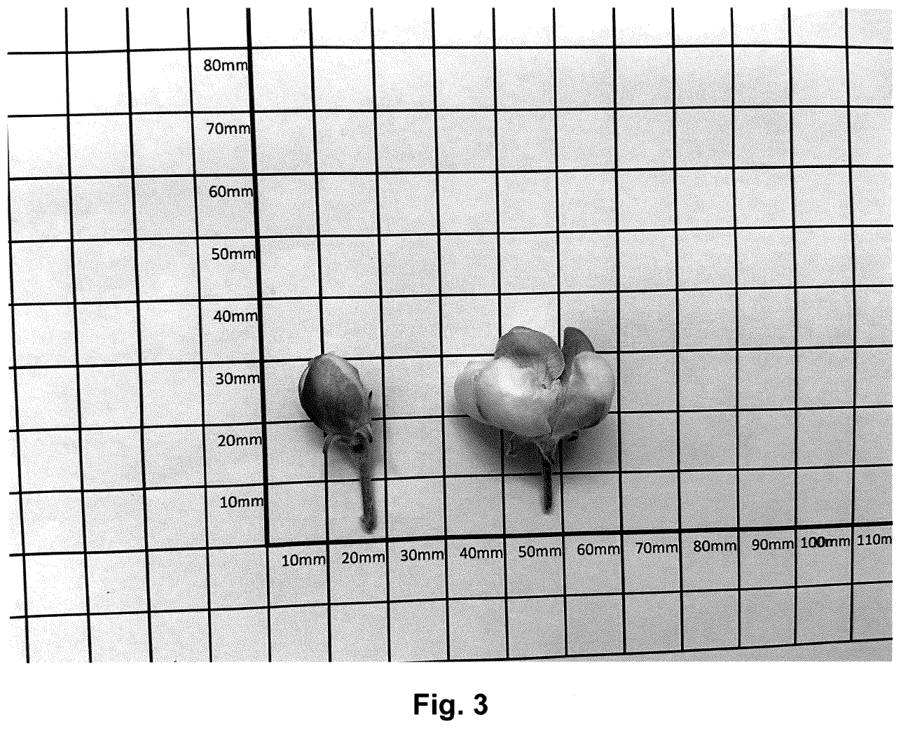

### DETAILED DESCRIPTION

The following is a description of the new variety with color terminology in accordance with The Royal Horticultural Society Color Charts (R.H.S.C.C.) fifth edition.

The specimens described were grown at Lleida, Catalonia, Spain. The observations were made over several seasons from 2014-2018 season on 5-9 year old trees.
Tree: Ramified growth habit, approximately 3.46 m tall and 1.24 m wide; spreading habit; bearing on spurs and long shoots; medium vigor on M9 rootstock, managed under standard orchard practice.
Trunk: Measurements were taken 20 cm from the graft union. Trunk circumference approximately 17.5 cm; color grey 199D; bark texture both smooth and rough texture.
One year old shoot: Approximately 25.6 cm long, thin thickness with straight growth; color on the sunny side near light brown 166A; average internode length 3.68 cm; number of lenticels were sparse, elongated in shape and light yellow 158B and approximately 0.62 mm in width and 1.7 mm in length.
Leaves: Average length 89.84 mm; average width 54.08 mm at the widest point; upper side of the blade near dark green N137A; underside near brown green 147B with weak pubescence; basal shape obtuse; apex shape right angled; margin of the leaves serrate; the attitude in relation to the shoot upwards. Vegetative bud burst early March.
Petiole: Average length 26.93 mm; average width 1.93 mm.
Stipule: Average length 6.06 mm; average width 0.44 mm; shape short and asymmetric; color dark green 136A on the upper side; under side color of the stipules medium brown green 147C.
Flowers: Color at balloon stage medium purple red, near 63A; diameter of the open flower approximately 27.2 mm; petals were not touching, and there were six flowers per cluster; time of beginning of flowering late March, followed by full bloom on the approximately 6th April.
Petals: Five petals per flower with a rounded shape; average length 14.6 mm; average width 11.75 mm; with a truncated base; lower surface coloration light violet 77D with near purple veins 72A.
Stamen: Average of 20 per flower. Filaments near white 155C and average length 8.56 mm. Anthers average length of 2.16 mm; average width 1.47 mm; color near light yellow 11B. Pollen color near yellow 3A. Anther position below stigma.
Carpel: Average length 12.1 mm; exterior color near light green 149C.
Sepals: Five sepals per flower; overall shape of an acute and curved triangle; smooth margin; average length 5.4 mm; average width 2.9 mm; color near green 144A.
Fruit size: Small; average width 68.31 mm; average height 58.27 mm; average weight 135.96 g.
Shape: Conic; position of maximum diameter slightly towards stalk; crowning at calyx end absent to weak. Eye basin average depth 12.196 mm; average width 23.17 mm.
Skin: Chewy texture; ribbing absent or weak; bloom of skin absent or weak; moderate; background color whitish yellow 11B; number of lenticels medium; size of lenticels medium.
Over color: Approximately 80% of skin surface; near dark purple red 181A; pattern of overcolor flushed, striped and mottled width of stripes medium; large amount of russet around stalk cavity; amount of russet on cheeks absent or small; area of russet around eye basin absent or small;
Stalk: Average length 19.21 mm; average width 2.83 mm; curvature medium. Average depth of stalk cavity 13.446 mm; average width 28.274 mm.
Flesh: Medium, average fruit firmness 6.2 kgf; very juicy; white 155C.
Flavor: Sweet; aroma sweet apple; soluble solids 13.1%.
Seeds: Five locules; medium seed size; average length 9.598 mm; average width 5.184 mm; color near dark brown 200C.
Harvest: Late July—early August.
Use: Dessert. Adapted to hot climate growing conditions. Less sensitive to sunburn and cracking than ‘Gala (Baigent)’.
Keeping quality: Excellent, up to 6 months at 0.5° C.+1 week shelf life.

1. A new and distinct variety of apple tree named ‘HOT81A1’ substantially as illustrated and described herein.
